SEQUENCE LISTING (2 sequences)

SEQ ID NO: 1 - protein, 26 aa, Artificial Sequence
  Description of Artificial Sequence  Synthetic Peptide
SAGFDFSFLPQPPQEKAHDGGRYYRA

SEQ ID NO: 2 - protein, 13 aa, Artificial Sequence
  Description of Artificial Sequence  Synthetic Peptide
EYDSYDVKSGVAC

(19) United States
(12) Patent Application Publication
(10) Pub. No.: US 2005/0232891 A1
(43) Pub. Date: Oct. 20, 2005

(54) Methods of treating skin with diphosphonate derivatives

(75) Inventors: Stephen J. Moloney, San Antonio, TX (US); Albert A. Jecminek, San Antonio, TX (US)

Correspondence Address: Steven L. Highlander, Esq.;FULBRIGHT & JAWORSKI L.L.P., Suite 2400, 600 Congress Avenue, Austin, TX, 78701, US

(73) Assignee: ILEX Products Inc.

(21) Appl. No.: 10/825,703

(22) Filed: Apr. 16, 2004

Related U.S. Application Data
(63) Continuation of application No. PCT/US02/32959, filed on Oct. 16, 2002.
(60) Provisional application No. 60/330,230, filed on Oct. 18, 2001.

(51) Int. Cl. A61K 31/663; A61K 31/665; A61K 7/075; A61K 7/08

(52) U.S. Cl. 424/70.23; 514/100; 514/102

(57) ABSTRACT

The present invention provides for the administration of aryl-substituted 1,1-diphosphonate compounds to increase the dermal collagen content of skin and effect improvement in photoaged skin, chronologically aged skin and in skin of postmenopusal women and patients suffering from osteoporosis.

### FIELD OF THE INVENTION

The present invention relates generally to the treatment of photoaged and chronological aged skin. More specifically, the invention relates to the administration of aryl-substituted 1,1-diphosphonate compounds to stimulate the expression of dermal collagen and for the effacement of wrinkles and thickening of skin.

### BACKGROUND OF THE INVENTION

Collagen is a major structural component of the dermis and is responsible for the strength and resilience of skin. The dermal extracellular matrix is composed primarily of type I collagen with lesser amounts of type III collagen (Smith et al., 1986). Alterations in dermal collagen have been causally related to the visible changes of skin associated with aging (Griffiths et al., 1993).

The aging process of skin can be sub-divided into photoaging and chronological or intrinsic aging (Yarr and Gilchrest 1998; Gilchrest, 1990). Photoaged skin is characterized by wrinkles, laxity, leathery appearance and changes in pigmentation (Leyden, 1990). A reduction in dermal collagen in sun-exposed skin is believed to be an etiological component of wrinkles (Lavker, 1979; Kligman, 1984). Alterations in photoaged skin include reduced levels of type I and type III collagen precursors (Talwar et al., 1995) and reduced levels of collagen crosslinks (Yamauchi et al., 1991). In addition, the clinical severity of photodamage in human skin has been shown to correlate with the decrease in dermal type I collagen (Griffiths et al., 1993).

Improvements in the visible signs of photoaging affected by chemical and physical treatments have been associated with an increase in dermal collagen. Retinoic acid-induced effacement of wrinkles has been correlated with increased dermal collagen synthesis (Chen et al. 1992; Griffiths et al, 1993). Glycollic acid skin peeling improves the appearance of photodamaged skin and glycollic acid increases the synthesis of collagen both in vitro (Kim & Won, 1998) and the expression and synthesis of collagen in vivo (Kim et al., 1998; Bernstein et al., 2001). Clinical improvement of photodamaged skin following physical abrasion also correlates with synthesis of collagen types I and III (Nelson et al, 1994; Nelson et al., 1996).

In regard to chronological aging, dermal collagen peaks about the third decade of life and then deceases at approximately one percent per year in both men and women. Skin thickness parallels this change in men, but in women skin thickness remains constant until menopause, after which skin thickness decreases (Arho, 1972; Shuster & Bottoms, 1963; Shuster et al., 1975). As in skin, type I collagen represents a major structural protein in bone and it been hypothesized, with the possible exception of pre-menopausal women, that age-related declines in bone density and skin thickness may be directly correlated (Whitmore & Levine, 1998). A correlation between bone density and skin thickness have been shown in post-menopausal women (Gruber et al, 1995; Gruber et al, 1995(b)). Further, skin collagen content and bone density have also been shown to decline in parallel in women, with decreases in the fourth decade and after menopause (Castelo-Branco et al., 1994). Estrogen replacement in postmenopausal women increases both skin collagen and skin thickness (Brincat et al, 1987; Casetlo-Branco et al., 1992). In addition, patients with osteoporosis have been shown to have a reduced skin collagen content (Black et al, 1970).

Many of the current treatments for photoaging are irritating. Hormone replacement therapies for postmenopausal women are also associate with potential contraindications. Thus, despite advances in the art, there is a need for safe and effective treatments for improving the visible effects of photoaged skin and increasing skin thickness in chronologically aged skin, especially in postmenopausal women, and in subjects suffering from osteoporosis.

### SUMMARY OF THE INVENTION

The present invention provides for the topical use of aryl-substituted 1,1-diphosphonate compounds to stimulate the expression of dermal collagen. In preferred embodiments, these well-tolerated compounds are applied to subjects with photoaged skin to effect the effacement of wrinkles or are applied to the skin of subjects who are postmenopausal or suffer from osteoporosis.

One aspect of the invention provides for a method for increasing the expression of dermal collagen in skin comprising the administration of an effective amount of a diphosphonate of the formula (1): 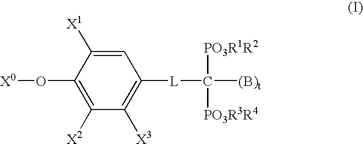
wherein X⁰ is H, an alkyl group having from 1 to 4 carbon atoms; X¹, X² and X³ are identical or different and are H, a straight or branched alkyl or alkoxy group having from 1 to 8 carbon atoms; R¹, R², R³ and R⁴ are identical or different and are H, a straight, branched or cyclic alkyl group comprising from 1 to 8 carbon atoms, or R¹, R², R³ and R⁴ may form an alkylidenedioxy ring comprising from 2 to 8 carbon atoms; B is H, or an alkyl group having from 1 to 4 carbon atoms; t is 0 or 1 with the proviso that when t is 1 then L is —CH═CH—CH₂—, —(CH₂)ₙ—, —O(CH₂)ₙ—, —S—, —SO₂—, —S(CH₂)ₙ—, —SO₂ (CH₂)ₙ—, where n is an integer from 1 to 7 and when t is 0 then L is —(CH═CH)ₖ—(CH₂)_{d} —CH═ where k is 0 or 1 and d is an integer from 0 to 4.

In preferred embodiments, X¹ and X² are the same and are both tert-butyl, X⁰ is H, L is CH₂, B is H, t is 1 and R¹, R², R³ and R⁴ are isopropyl. In other embodiments R¹, R², R³ and R⁴ are the same or different and are selected from hydrogen, methyl, ethyl, n-propyl, isopropyl, n-butyl, s-butyl and tert-butyl. In a preferred embodiment, the compound of formula (I) is tetraisopropyl 2-(3,5-di-tert-butyl-4-hydroxyphenyl)-ethylidene-1,1-diphosphonate (Apomine).

In preferred embodiments of the invention, the compound of formula (I) is applied topically to photoaged skin, postmenopausal skin or to the skin of a subject with osteoporosis. The invention provides that the increase of dermal collagen expression may be an increase in expression of collagen type I and/or collagen type III.

Other aspects of the invention provide for a method for increasing skin thickness and a method for retarding and/or reversing the formation of fine lines and wrinkles, comprising the administration, preferably by topical application, of an effective amount of a diphosphonate of formula (I). A further aspect of the present invention is a topical pharmaceutical composition comprising a pharmaceutical topical carrier and an amount of a compound of formula (I) effective to stimulate the expression of dermal collagen when applied to skin. In various embodiments, the composition may further comprise an additional component selected from an inhibitor of matrix metalloproteinase production or activity, a retinoid, an alpha hydroxy acid or derivative thereof, an anti-oxidant, a radical scavenging agent, or an anti-inflammatory agent, or a mixture thereof.

### DETAILED DESCRIPTION OF THE INVENTION

### I. ARYL-SUBSTITUTED 1,1-DIPHOSPHONATE COMPOUNDS

The present invention provides for the administration of aryl-substituted 1,1-diphosphonate compounds of formula (I) to increase the dermal collagen content of skin and effect improvement in photoaged skin, chronologically aged skin and in skin of postmenopausal women and patients suffering from osteoporosis.

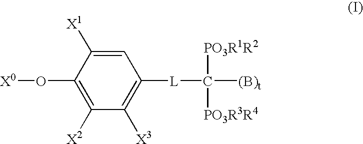

Examples of groups X⁰, X¹, X² and X³ include, but are not limited to, hydrogen, straight or branched alkyl groups and alkoxy groups having from 1 to 8 carbon atoms, more particularly from 1 to 4 carbon atoms. In preferred embodiments, X¹ and X² are independently methyl, ethyl, n-propyl, isopropyl, sec-butyl, methoxy and ethoxy groups, and in more preferred embodiments are independently t-butyl. In a preferred embodiment, X³ is hydrogen and X¹ and X² are t-butyl.

The substituents represented by R¹, R², R³ and R⁴ include hydrogen and straight, branched or cyclic alkyl groups comprising from 1 to 8 carbon atoms. In various embodiments R¹, R², R³ and R⁴ may be independently hydrogen, methyl, ethyl, n-propyl, isopropyl, n-butyl, sec-butyl and t-butyl. In a preferred embodiment, R¹, R², R³ and R⁴ are independently ethyl or isopropyl. In other embodiments, R¹, R², R³ and R⁴ may form an alkylidenedioxy ring comprising from 2 to 8 carbon atoms.

In a preferred embodiment, the compound of formula (I) is Apomine (tetraisopropyl 2-(3,5-di-t-butyl-4-hydroxyphenyl)ethynilidene-1,1 diphosphate).

The compounds of formula (I) may be prepared by methods disclosed in U.S. Pat. No. 5,043,330 and 5,204,336, both herein incorporated by reference.

### II. PERCUTANEOUS ABSORPTION

Diphosphonic acid derivatives have been disclosed to be surprisingly readily conveyed through skin (U.S. Pat. No. 5,133,972, herein incorporated by reference). Increasing the lipophilicity of diphosphonic acids by forming esters derivates, as in the diphosphonate compounds of the present invention, will typically further increase the percutaneous absorption. In concert with these observations, Apomine has been shown to absorbed after topical application, wherein cutaneous levels will be in excess of those required for increasing collagen expression (Examples 1 and 3 as disclosed herein).

III. DERMAL COLLAGEN

The predominate collagen in the human dermis is collagen Type I, accounting for approximately 85% of the total, with collagen type III accounting for approximately 10%. (Smith et al., 1986). Procollagen is synthesized by dermal fibroblasts and are assembled as a triple helix, comprising a number of distinct alpha chains, flanked by propeptides at the carboxy and amino terminals. After secretion into the dermal extracellular matrix, procollagen is cleaved by amino- and carboxy-terminal proteases to form mature collagen. The term pN collagen refers to an intermediate form of procollagen that has lost the carboxypropetide and pC collagen refers to an intermediate that has lost the aminopropeptide. Apomine increases the expression of a number of collagens including collagen type I alpha 2 and procollagen type III (Example 1 herein disclosed).

### IV. TOXICITY

Both animal toxicity studies and human clinical studies with bisphosphonate esters have revealed that they are well tolerated in man. For instance, results from a recent clinical study showed that patients receiving a dose of 75 mg/m² of Apomine twice a day orally for 14 days experienced no adverse effects (Alberts et al., 2001).

### V. FORMULATIONS AND ADMINISTRATION

The administration of the diphosphonate compounds of the present invention is contemplated to be primarily via a topical route. A variety of formulations for the topical application of the compounds of the present invention are contemplated, wherein the formulations contain the diphosphonate compounds of formula (I) in a concentration effective to stimulate the expression of collagen in the dermis of the treated skin. The topical formulation of the present invention may contain contains a diphosphonate compound in a total concentration of about 0.0001 to about 5 percent by weight of formulation, which may be about 0.001, or about 0.01, or about 0.1, or about 0.5, or about 1.0, or about 2.0 percent by weight of formulation.

The compositions and methods of the present invention further contemplate topically administrating a diphosphonate of the present invention in conjunction with effective amounts of other agents beneficial for the treatment and/or prevention of the cutaneous effects of photoaged and chronologically aged skin. Such agents include inhibitors of UVB-induced matrix metalloproteinase production or activity, e.g., as disclosed in U.S. Pat. No. 5 837,224 (herein incorporated by reference), retinoic acid or retinoid compounds, e.g., as disclosed in U.S. Pat. Nos. 4,877,805 and 5,124,356 both (herein incorporated by reference), alpha hydroxy acids and hydroxy acid derivatives, e.g., as disclosed in U.S. Pat. No. 5,686,489 (herein incorporated by reference), anti-oxidants and radical scavenging agents, e.g., as disclosed in U.S. Pat. Nos. 5,384,115 and 5,739,156 (both herein incorporated by reference), or anti-inflammatory compounds, e.g., as disclosed in U.S. Pat. No. 5,709,847, herein incorporated by reference. The diphosphonates of the present invention may also be administered in combination with compounds suitable to decrease epidermal proliferation and/or treat UV-induced pre-malignant lesions, e.g., difluoromethylornithine.

In accordance with the method of the present invention, the components of a combination of the diphosphonates compounds of the present invention and other agents beneficial for the treatment and/or prevention of the cutaneous effects of photoaged and chronologically aged skin can be administered separately at different times during the course of administration or concurrently in divided or single combination forms. According to the instant invention, the term administering is to be understood as embracing all such regimes of simultaneous or alternating treatment and the scope of combinations of the compounds of this invention and other agents includes in principle, any combination useful for the treatment and/or prevention of the cutaneous effects of photoaged and chronologically aged skin.

Suitable topical vehicles and components for use with the formulations of the present invention are well known in the art. Such vehicles include water; organic solvents such as alcohols (such as ethanol); glycols (such as propylene glycol); aliphatic alcohols (such as lanolin); mixtures of water and organic solvents and mixtures of organic solvents such as alcohol and glycerin; lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile); hydrocarbon-based materials such as microsponges and polymer matrices; stabilizing and suspending agents; emulsifying agents; and other vehicle components that are suitable for administration to the skin, as well as mixtures of these components and those otherwise known in the art. The vehicle can further include components adapted to improve the stability or effectiveness of the applied formulation, such as preservatives, antioxidants, skin penetration enhancers and sustained release materials. Examples of such components are described in the following reference works hereby incorporated by reference: Martindale-The Extra Pharmacopoeia (Pharmaceutical Press, London 1993) and Martin (ed.), Remington's Pharmaceutical Sciences.

The choice of a suitable vehicle will depend on the particular physical form and mode of delivery that the formulation is to achieve. Examples of suitable forms include lotions, emulsions, solids and semisolids such as gels, foams, pastes, creams, ointments, “sticks,” powders and the like. These formulations, may also contain solvents, emulsifiers, moisturizers, emollients, fragrances, dyes/colorants, preservatives and other active ingredients that increase or enhance the efficacy of the final product.

Suitable emulsifiers for use in the formulations of the present invention include, but are not limited to ionic emulsifiers; behentirmonium methosulfate, cetearyl alcohol; non-ionic emulsifiers like polyoxyethylene oleyl ether, PEG-40 sterate, ceteareth-12, ceteareth-20, ceteareth-30, ceteareth alcohol, PEG-100 stearate, glyceryl stearate, or combinations or mixtures thereof. Suitable viscosity adjusting agents include, but are not limited to protective colloids or non-ionic gums such as hydroxyethylcellulose, xanthan gum, magnesium aluminum silicate, silica, microcrystalline wax, beeswax, paraffin, and cetyl palmitate, or combinations or mixtures thereof. Suitable solvents include, but are not limited to water, ethanol, butylene glycol, propylene glycol, isopropyl alcohol, isoprene glycol, and glycerin. In addition, combinations or mixtures of these solvents can be used in the formulations of the present invention. Suitable surfactants include, but are not limited to nonionic, amphoteric, ionic and anionic surfactants. For example, dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate are contemplated for use with the formulations of the present invention. In addition, combinations or mixtures of these surfactants can be used in the formulations of the present invention. Suitable preservatives include, but are not limited to antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. In addition, combinations or mixtures of these preservatives can be used in the formulations of the present invention. Suitable moisturizers include, but are not limited to lactic acid and other hydroxy acids and their salts, glycerin, proplyene glycol, and butylene glycol. Suitable emollients include lanolin alcohol, lanolin, lanolin derivatives, cholesterol, petrolatum, isostearyl neopentanoate and mineral oils. In addition, combinations or mixtures of these moisturizers and emollients can be used in the formulations of the present invention. Suitable active ingredients in addition to the diphosphonate compounds of the present invention include, but are not limited to alpha hydroxy acids, sunscreens, vitamins and minerals.

The formulations of the present invention may comprise a penetration enhancer. Methods for enhancing the local skin penetration of pharmacologically active agents are known in the art (See, e.g., Finnin & Morgan, 1999; Hadgraft, 1996). Examples of suitable enhancers include, lecithin, ethanol, propylene glycol, water, sodium oleate, leucinic acid, oleic acid, capric acid, sodium caprate, lauric acid, sodium laurate, neodecanoic acid, dodecyl-amine, cetryl lactate, myristyl lactate, lauryl lactate, methyl laurate, phenyl ethanol, hexa-methylene lauramide, urea and derivatives, dodecyl N, N-dimethylamino acetate, hydroxyethyl lactamide, phyophatidylcholine, sefsol-318 (a medium chain glycer-ide), isopropyl myristate, isopropyl palmitate, several surfactants, including poly-oxyethylene (10) lauryl ether (Brij 361 R), diethyleneglycol lauryl ether (PEG-2-L), laurocapram (Azone; 1,1-dodecylazacycloheptan-2-one), acetonitrile, 1-decanol, 2-pyrrolidone, N-methylpyrrolidone, N-ethyl-1-pyrrolidone, 1-methyl-2-pyrroli-done, 1-lauryl-2-pyrrolidone, sucrose monooleate, dimethylsulfoxide (DMSO) about 80% concentration required, decylmethylsulfoxide enhances primarily polar or ionic molecules (soluble in ethanol), acetone, polyethylene glycol 100-400 MW, dimethylacetamide, dimethylforamide, dimethylisosorbide, sodium bicarbonate, various n-C₇₋₁₆-alkanes, mentane, menthone, menthol, terpinene, D-terpinene, dipen-tene, n-nonalol and limonene.

Additional ingredients that may be included in the formulation of the present invention include, but are not limited to, abrasives, absorbents, anti-caking agents, anti-foaming agents, anti-static agents, astringents (e.g., witch hazel, alcohol and herbal extracts such as chamomile extract), binders/excipients, buffering agents, chelating agents, film forming agents, conditioning agents, opacifying agents, pH adjusters and protectants. Examples of each of these ingredients in topical product formulations, can be found in publications by The Cosmetic, Toiletry, and Fragrance Association (CTFA). See, e.g., CTFA Cosmetic Ingredient Handbook, 2nd edition, eds. John A. Wenninger and G. N. McEwen, Jr. (CTFA, 1992).

Controlled release vehicles can also be used to administer the compounds of the present invention. The technology and products in this art are variably referred to as controlled release, sustained release, prolonged action, depot, repository, delayed action, retarded release and timed release; the words “controlled release” as used herein is intended to incorporate each of the foregoing technologies. Numerous controlled release vehicles are known, including biodegradable or bioerodable polymers such as polylactic acid, polyglycolic acid, and regenerated collagen. Known controlled release drug delivery devices include creams, lotions, tablets, capsules, gels, microspheres, and liposomes. Transdermal formulations, from which active ingredients are slowly released are also well known and can be used in the present invention.

The present invention also encompasses oral administration of compounds of formula (I). Tablets and other solid or liquid oral dosage forms can be prepared (e.g., in standard fashion) from the compounds of formula (I) and a pharmaceutically acceptable solubilizer, diluent or carrier. Examples of solubilizers, diluents or carriers include sugars such as lactose, starches, cellulose and its derivatives, powdered tracaganth, malt, gelatin, talc, stearic acid, magnesium stearate, calcium sulfate, vegetable oils, polyols such as glycerol, propyleneglycol and polyethyleneglycols, alginic acids and alginates, agar, pyrogen free water, isotonic saline, phosphate buffered solutions, and optionally other pharmaceutical excipients such as disintegrants, lubricants, wetting agents such as sodium lauryl sulfate, coloring agents, flavoring agents and preservatives, etc.

Capsules can be of the hard or soft variety and can contain the active compound in solid, liquid or semisolid form. Typically such capsules are formed from gelatin or an equivalent substance and can be coated or uncoated. If it is desired to delay the release of the active compound until the capsule has passed through the stomach and into the intestine, the capsule can be provided with a pH sensitive coating adapted to dissolve at the pH found in the duodenum or ileum. Examples of such coatings include the Eudragits, the uses of which are well known.

Oral administration of the compounds of formula (I) may be combined with topical application of compounds of formula (I). For example, an initial oral dose or doses may be followed by a maintenance regimen utilizing topical administration.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Stimulation of Collagen Expression

HepG2 cells were grown in Dulbecco's modified Eagle medium (DMEM) supplemented with 10% FBS. Cells were harvested when they were about 80-90% confluent and were directly subjected to RNA isolation. Treated cells were incubated with 1 μM Apomine and control cells were incubated with a volume of ethanol used as vehicle for the Apomine.

DNA microarray slides used in this study were fabricated in the microarray core facilities at the Arizona Cancer Center (Calaluce et al., 2001). Each slide has 5760 spots divided into 4 blocks. Each block contains the same 8 ice plant genes from Mesembryanthemum crystallinum and 23 different housekeeping genes as references for data normalization. Overall, each slide has 5289 unique human cDNA sequences.

Poly(A)⁺ RNA was directly isolated from cell pellets using the FastTrack 2.0 Kit (Invitrogen, Carsbad, Calif.) following the instruction manual provided by the manufacturer. Each RNA sample was inspected by denaturing agarose gel electrophoresis to ensure the quality of the preparation. Labeling and purification of cDNA probes were carried out using the MICROMAX direct cDNA microarray system (NEN Life Science Products, Boston, Mass.). Two to four micrograms of the poly(A)+ RNA samples were used for each labeling. Probes for Apomine treated HepG2 cells were labeled with Cyanine 5 (Cy5) and probes for HeLa cell was labeled with Cyanine 3 (Cy3). For investigation of control Hep2G cells, these cells were labeled with Cy3 and probes for HeLa cells were labeled with Cy5. Purified cDNA probes were dried and dissolved in 15 μL of Hybridization Buffer (included in the MICROMAX direct cDNA microarray system kit). The probes were then denatured by heating at 95° C. for 2 min and applied to the array area of a pre-denatured microarray slide. The microarray slide was covered with a 22 cm×22 cm slide cover slip and incubated in a HybChamber (GeneMachines, San Carlos, Calif.) at 62° C. for overnight. On the second day, the slide was washed in 0.5×SSC, 0.01% SDS for 5 min, 0.06×SSC, 0.01% SDS for 5 min, and 0.06×SCC for 2 min. Finally, the slide was dried by spinning at 500 g for 1 min and scanned in a dual-laser (635 nm for red fluorescent Cy5 and 532 nM for green fluorescent Cy3) microarray scanner (GenePix 4000, Axon Instruments, Foster City, Calif.).

Fluorescence intensities for both dyes (Cy3 and Cy5) and local background subtracted values for individual spots were obtained using the GenePix 4000 microarray scanner and accompanying software (Axon Instruments, Foster City, Calif.). The data were imported into Microsoft Excel spreadsheets for analysis. Defective spots, i.e., ones that are sub-standard on the scanned image or have negative background subtracted values, were first filtered. To minimize the effects of measurement variations introduced by artificial sources during experiments, only spots that had significant signals in both channels were included. The determination of this significance is based on signal intensities of non-homologous ice plant genes. Generally, if the signal intensity of a spot is less than the average of ice plant spots, the signal was considered as non-significant. In this analysis the significance cutoff for signal to background ratio as 1.4 was determined empirically. Thus, a spot was excluded in further analysis if it has a signal to background ratio less than 1.4 in both channels. For each spot, the median of ratios (the median of the pixel-by-pixel ratios of pixel intensities that have the median background intensity subtracted) was used in subsequent analysis. Spots representing housekeeping genes were used to normalize the entire slide so that all slides can be compared directly. For each pancreatic cell line, at least two hybridizations were carried out. The average of median ratios from replicates was calculated for each spot.

To ensure that the exact same reference samples were used for all necessary experiments, a HeLa cell mRNA pool was used as a universal reference for microarray hybridizations. In other words, the Cy5 labeled probes for Apomine treated HepG2 cells mixed with Cy3 labeled probes for Hela cells and hybridized to one slide to obtain the ratio of Apomine treated HepG2 cells versus Hela cells. On the other hand, Cy5 labeled HeLa cell probes were mixed with Cy3 labeled control HepG2 cell probes and hybridized to a slide to obtain the ratio of HeLa cell versus control HepG2 cells. Each slide was normalized by the housekeeping genes, so that errors caused by hybridization differences from slide to slide are minimized. The two ratios were then multiplied to generate the ratio of Apomine treated cells to control cells. Finally, the ratios were taken a log2 transformation and the standard deviations from the mean were then calculated from these log2-ratios for each cell line. A 2.0 standard deviations cutoff was used for the determination of expression outliners.

The result regarding expression of collage genes are shown in TABLE 1. Apomine treatment resulted in over a 6-fold increase in the expression of type III procollagen and a 1.65-fold increase in type I collagen (alpha 2) expression.

TABLE 1

| Gene | Fold induction |
|---|---|
| Procollagen, type III | 6.49 |
| Collagen, type I, alpha 2 | 1.65 |
| Collagen, type II, alpha 1 | 5.50 |
| Collagen, type IV, alpha 4 | 1.70 |
| Collagen, type V, alpha 1 | 1.42 |
| Collagen, type VI, alpha 2 | 2.40 |
| Collagen, type VI, alpha 3 | 1.16 |
| Collagen, type IX, alpha 3 | 1.40 |
| Collagen, type XI, alpha 1 | 2.66 |

### Example 2

### Measurement of Apomine

Plasma concentrations of Apomine are measured with a Hewlett Packard gas chromatograph using a nitrogen phosphorus detector and HP-5 15 m×0.32 mm column. Alberts et al., 2001). Apomine is extracted from plasma with methyl t-butyl ether (MTBE). To 250 μl of plasma sample, 10 μl of a methanolic solution of the internal standard (n-propyl phoposhonate analog of Apomine) at a final concentration of 4 μg/ml are added and mixed by vortexing. The compound and internal standard are extracted with 500 μl MTBE and 30 sec vigorous shaking. The organic layer obtained by centrifugation (5 min at 13,000 rpm) is dried under nitrogen and reconstituted in 250 μl MTBE of which 2 μl is injected with an autoampler for analysis. A calibration curve is established with plasma from untreated mice spiked with different concentrations of Apomine. The calibration curve is used to interpolate the concentrations of Apomine in the samples using the peak area ratio values.

### Example 3

### Percutaneous Absorption of Apomine

Apomine (1 mg) was topically applied to five to six week old female Swiss nude (nu/nu) mice by application of 20 μl of a 50 mg/ml solution of Apomine in acetone. Treatment was continued daily for 24 days. Three hours after the last application the mice were sacrificed and plasma Apomine levels were measured. The plasma concentration was 1.12±0.10 μg/ml plasma (n=6), which is 2 μM Apomine. It is to be expected that the local concentration of Apomine achieved in skin will be in excess, and possibly in far excess, of the concentration of Apomine achieved in the plasma. Thus, the skin level will be in excess of the 1 μM concentration of Apomine that resulted in increased collagen expression in Hep2 cells.

### Example 4

### Clinical Trial: Amelioration of Photodamage

Volunteers are recruited aged 35-70 who present with wrinkles on the face of moderate to sever intensity as defined by means of a photograder technique (Griffths et al., 1992). Patients with suspected skin cancers, facial dermatoses, known allergies to ingredients of the formulation, of who had used topical retinoids, chemical peelings, or an abrasive substance on the skin within 45 days before entry into the study are excluded. Pregnant and nursing women and patients who planned to use tan or phototherapy were also excluded. Volunteers are randomly assigned to one of two treatments groups, topical formulation containing a diphosphonate compound of the present invention and a formulation vehicle control. Patients apply 0.5-0.75 gm of formulation on the entire surface of the face daily for the course of the study. Patients are also instructed to use a sunscreen (SPF 20) on the test area before outdoor activities.

A. Assessment of Wrinkles

At time 0, 1, 3 and 6 months, wrinkling is measured by clinical assessment and/or skin surface profilometry. A clinical assessment by an investigator measures coarse and fine wrinkling scored on a scale of 0 to 4 according to intensity and deepness of wrinkles with 0 equating to no wrinkles and 4 equating to severe wrinkles. Skin surface profilometry by means of computerized image analysis of silicone rubber skin replicas is performed as described by Grove et al. (1991) and Corcuff et al. (1988). Skin surfaces are molded using a silicone material with adhesive rings used to delineate sample sites and specimen orientation. Replicas are analyzed in a randomized fashion by optical profilometry wherein the degrees of wrinkling and roughness are calculated.

B. Biochemical, Histochemical and Immunohistochemical Analyzes

Biochemical, histochemical and immunohistochemical analyzes can be undertaken on skin biopsy samples. In that patients may not be receptive to facial skin biopsies, clinical studies can also be undertaken with patients that present with clinical evidence of sun damage and wrinkling on the posterior forearm. Such studies can use pair controls with left and right arms as described in Bernstein et al. (2001).

Collagen Type I mRNA: Total RNA from frozen skin biopsy specimens is isolated as described by Bernstein et al, (1995). RNA is analyzed by Northern hybridization with a ³²P-labelled human type I collagen cDNA probe (Bernstein et al., 2001). The preparation of and localization of type I collagen RNA probe has been described by Katakam et al., (1992). A 705-base pair fragment (2316 to 3021 base pairs) of original cDNA Hf677 clone of collagen type 1 proalpha 1 is subcloned into the EcoRI site of pGEM37 (Promega, Madison, Wis.). Following linearization with Xho I or BamHI (Gibco-BRL Lifetechnologies, Gaithersburg, Md.), in vitro transcription is carried out using a digoxigenin RNA labeling kit (Boehringer Mannheim, Indianapolis, Ind.) to generate antisense and sense RNA probes. [³²P]cDNA-mRNA hybrids are visualized by autoradiography and the steady-state levels of mRNA are quantitated by scanning densitometry. Collagen mRNA levels can be standardized to 7S rRNA levels in the same RNA sample.

Collagen Immunohistochemistry: Formalin fixed frozen sections are prepared by routine procedures and used for immunohistochemcial analyzes. Collagen type I and Type III specific antibodies can be prepared by using selected sequences from for the telopeptide (non-triple helical segments on the carboxy and amino terminal ends present in the mature collagens) as immunogens. The collagen I alpha 1 telopeptide sequence SAGFDFSFLPQPPQEKAHDGGRYYRA (SEQ ID NO: 1) and collagen type III alpha 1 telopetide sequence EYDSYDVKSGVAC (SEQ ID NO:2) can be used as immunogens to raise polyclonal antibodies suitable for immunohistochemistry (Bernstein et al., 1996). In addition, rat anti-human procollagen I (amino terminal of alpha 1 and alpha 2 chains) monoclonal antibody 1912 can be obtained from Chemicon International (Temecula, Calif.) and anti-human procollagen type I c-peptide can be obtained from PanVera Corp. (Madison Wis.). Antibodies are visualized using fluorescent or enzymatic techniques routinely used in the art. The relative degree of immunostaining can be assessed with a semiquantitative 0 to 4 scale where 0=absent. 1=low, 2=moderate, 3=high and 4=maximum.

Histochemical Analyzes: Formalin-fixed sections are stained with Masson's trichrome for histological assessment of dermal collagen and hematoxylin and eosin for overall general investigation.

### Example 5

### Clinical Trial: Treatment of Postmenopausal Skin

Female post-menopausal women are recruited. Volunteers with suspected skin cancers, dermatoses associated with the treatment site, or known allergies to ingredients of the formulation are excluded. Patients apply 0.25-0.50 gm of a formulation containing a diphosphonate compound of the present invention on the surface of the posterior forearm of one arm and an equivalent amount of a vehicle control on the posterior forearm of the other arm daily for the course of the study. At time 0, 1, 3 and 6 months, skin thickness is measured. Skin thickness can be measured by skinfold calipers, ultrasound or radiography (Lawrence & Shuster, 1985; Dykes et al., 1976; Newton et al., 1984). Biochemical, histochemical and immunohistochemical analyzes can be undertaken on paired skin biopsy samples as described previously.

The present invention has been shown by both description and examples. The Examples are only examples and cannot be construed to limit the scope of the invention. One of ordinary skill in the art will envision equivalents to the inventive process described by the following claims that are within the scope and spirit of the claimed invention.

All of the composition and methods disclosed and claimed herein can be made and executed without undue experimentation in light of the present disclosure. While the compositions and methods of this invention have been described in terms of preferred embodiments, it will be apparent to those of skill in the art that variations may be applied to the composition and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference.

Alberts et al., Phase I pharmacokinetic trial and correlative in vitro phase II tumor kinetic study of Apomine (SR-45023A), a novel oral bisphosphonate anticancer drug, Clin. Cancer Res., 7:1246-50, 2001.

Arho, Skin thickness and collagen content in some endocrine, connective tissue and skin diseases, Acta Derm. Venerol. Suppl., 69:1-48, 1972.

Bernstein et al., Differential expression of versican and decorin genes in photoaged and sun-protected skin: comparison by immunohistochemical and Northern analyses, Lab. Invest., 72:662-69, 1995.

Bernstein et al., Long-term sun exposure alters the collagen of the papillary epidermis, J. Am. Acad. Dermatol., 34:209-18, 1996.

Bernstein et al., Glycolic acid treatment increased type I collagen mRNA and hyaluronic acid content of human skin, Dermatol. Surg. 27:429-33, 2001.

Black et al., Osteoporosis, skin collagen, and androgen, Brit. Med. J. 4:773-4, 1970.

Brincat et al., Skin collagen changes in postmenopausal women receiving different regimens of estrogen therapy, Obstet. Gyncol., 70:123-7, 1987.

Calaluce et al., Laminin-5-mediated gene expression in human prostate carcinoma cells. Mol. Carcinog. 30:119-29, 2001.

Castelo-Branco et al., Skin collagen changes related to age and hormone replacement therapy, 15:113-9, 1992.

Castelo-Branco et al., relationship between collagen and bone changes during aging, Maturitas, 18:199-206, 1994.

Chen et al., Effects of all-trans retinoic acid on UVB-irradiated and non-irradiated hairless mouse skin, J. Invest. Dermatol. 98:248-54, 1992.

Corcuff et al., Microrelief changes in chronically sun-exposed human skin., Photodermatology, 5:92-95, 1988.

Dykes et al., Measurement of dermal thickness with the Harpenden skinfold caliper, Arch. Dermatol. Res., 27:261-3, 1976.

Finnin & Morgan, Transdermal penetration enhancers: applications, limitations and potential, J. Pharm. Sci., 88:955-8, 1999.

Gilchrest, Skin aging and photoaging, Dermatol Nurs. 2:79-82, 1990.

Griffiths et al., A photonumeric scale in chronically sun-exposed human skin, Arch. Dermatol., 128:347-51, 1992.

Griffiths et al., Restoration of collagen formation in photodamaged human skin by tretinoin (retinoic acid), N. Engl. J. Med. 329:530-35, 1993.

Grove et al., Skin replica analysis of photodamaged skin after therapy with retinoin emollient cream., J. Amer. Acad. Dermatol., 25:231-37, 1991.

Gruber et al., Measuring skin thickness in perimenopausal women: Correlation with bone density and hormone parameters, Ultrschall Med., 16:22-4, 1995(a).

Gruber et al., Correlation of skinfold thickness with bone density and estradiol, FSH and prolactin level in a sample of 231 women, Wien Klin. Wochenschr, 107:622-5, 1995(b).

Hadgraft, Recent developments in topical and transdermal delivery, Eur. J. Drug. Metab. Pharmacokinet. 21:165-73, 1996.

Katakam et al., Localization of collagen (I) and collagenase mRNA by in situ hybridization during corneal wound healing after epikeratophakia or alkali burn, Jpn. J. Opthalmol., 36:10-22, 1992.

Kim et al., Increased in vivo collagen synthesis and in vitro cell proliferative effect of glycolic acid, Dermatol. Surg., 24:1054-58, 1998.

Kim & Won, The effect of glycolic acid on cultured human skin fibroblasts: cell proliferative effect and increased collagen synthesis, J. Dermatol. 25:85-89, 1998.

Kligman, The ultraviolet-irradiated hairless mouse: a model for photoaging, J. Am. Acad. Dermatol., 21:623-31, 1989.

Lavker, Structural alterations in exposed and unexposed aged skin, J. Invest. Dermatol., 73:59-66, 1979.

Lawrence & Shuster, Comparison of ultrasound and caliper measurements of normal and inflamed skin thickness, Br. J. Dermatol. 112:195-200, 1985.

Leyden, Clinical features of ageing skin, Br. J. Dermatol., 122: Suppl. 35:1-3, 1990.

Nelson et al., Clinical improvements following dermabrasion of photoaged skin correlates with synthesis of collagen I, Arch. Dermatol., 130:1136-1142, 1994.

Nelson et al., A comparison of wire brush and diamond fraise superficial dermabrasion for photoaged skin, J. Am. Acad. Dermatol., 34:235-243, 1996.

Newton et al., A comparison of pulsed ultrasound, radiography and micrometer screw gauge in the measurement of skin thickness, Curr. Med. Res. Opin. 2:113-8, 1984.

Shuster et al., The influence of age and sex on skin thickness, skin collagen and density, Br. J. Dermatol., 93:639-43, 1963.

Shuster & Bottoms, Senile degeneration of skin collagen, Clin. Sci. 25:639-43, 1975.

Smith et al., Collagen Types I, III and V in human embryonic and fetal skin. Am. J. Anat. 175:507-21, 1986.

Talwar et al., Reduced type I and type III procollagens in photodamaged adult human skin, J. Invest. Dermatol., 105:285-90, 1995.

Whitmore & Levine, Risk factors for reduced skin thickness and bone density: Possible clues regarding pathophysiology, prevention and treatment, J. Am. Acad. Dermatol., 38:248-55, 1998.

Yaar & Gilchrest, Aging versus photoaging: postulated mechanisms and effectors, J. Invest. Dermatol. Symp. Proc., 3:47-51, 1998.

Yamauchi et al., Collagen cross-linking in sun-exposed and unexposed sites of aged human skin, J. Invest. Dermatol. 97:938-41, 1991.

1. A method for increasing the expression of dermal collagen in skin comprising the administration of an effective amount of a diphosphonate of formula (I): 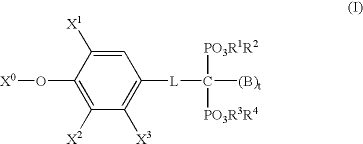
wherein X⁰ is H, an alkyl group having from 1 to 4 carbon atoms;
X¹, X² and X³ are identical or different and are H, a straight or branched alkyl or alkoxy group having from 1 to 8 carbon atoms,
R¹, R², R³ and R⁴ are identical or different and are H, a straight, branched or cyclic alkyl group comprising from 1 to 8 carbon atoms, or R¹, R², R³ and R⁴ may form an alkylidenedioxy ring comprising from 2 to 8 carbon atoms;
B is H, or an alkyl group having from 1 to 4 carbon atoms,
t is 0 or 1,
when t is 1 then L is —CH═CH—CH₂—, —(CH₂)ₙ—, —(CH₂)ₙ—, —s—, —SO₂—, —S(CH₂)ₙ—, —SO₂ (CH₂)ₙ—, where n is an integer from 1 to 7;
or when t is 0 then L is —(CH═CH)ₖ —(CH₂)_{d} —CH═ where k is 0 or 1 and d is an integer from 0 to 4:

2. The method of claim 1, wherein said administration is by topical application.

3. The method of claim 1, wherein X¹ and X² are the same and are both tert-butyl and X⁰ is H.

4. The method of claim 3, wherein L is CH₂, B is H and t is 1.

5. The method of claim 4, wherein R¹, R², R³ and R⁴ are isopropyl.

6. The method of claim 1, wherein R¹, R², R³ and R⁴ are the same or different and are selected from hydrogen, methyl, ethyl, n-propyl, isopropyl, n-butyl, s-butyl and tert-butyl.

7. The method of claim 6, wherein R¹, R², R³ and R⁴ are identical but are not H.

8. The method of claim 7, wherein R¹, R², R³ and R⁴ are isopropyl.

9. The method of claim 1, wherein said diphosphonate of formula (I) is tetraisopropyl 2-(3,5-di-tert-butyl-4-hydroxyphenyl)-ethylidene-1,1-diphosphonate.

10. The method of claim 1, wherein said collagen is collagen type I and/or collagen type III.

11. The method of claim 1, wherein said skin is photoaged skin.

12. The method of claim 1, wherein said skin is postmenopausal skin.

13. The method of claim 1, wherein said skin is the skin of a subject with osteoporosis.

14. The method of claim 1, further comprising administration of an effective amount of an inhibitor of matrix metalloproteinase production or activity, a retinoid, an alpha hydroxy acid or derivative thereof, an anti-oxidant, a radical scavenging agent, an anti-inflammatory agent, or a mixture thereof.

15. A method for increasing skin thickness comprising the administration of an effective amount of a diphosphonate of formula (I): 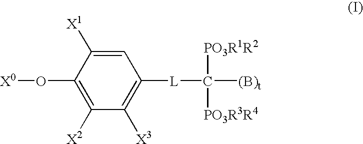
wherein X⁰ is H, an alkyl group having from 1 to 4 carbon atoms;
X¹, X² and X³ are identical or different and are H, a straight or branched alkyl or alkoxy group having from 1 to 8 carbon atoms,
R¹, R², R³ and R⁴ are identical or different and are H, a straight, branched or cyclic alkyl group comprising from 1 to 8 carbon atoms, or R¹, R², R³ and R⁴ may form an alkylidenedioxy ring comprising from 2 to 8 carbon atoms;
B is H, or an alkyl group having from 1 to 4 carbon atoms,
t is 0 or 1,
when t is 1 then L is —CH═CH—CH₂—, —(CH₂)ₙ—, —O(CH₂)ₙ—, —S—, —SO₂—, —S(CH₂)ₙ—, —SO₂ (CH₂)ₙ—, where n is an integer from 1 to 7;
or when t is 0 then L is —(CH═CH)ₖ —(CH₂)_{d} —CH═ where k is 0 or 1 and d is an integer from 0 to 4.

16. The method of claim 15, wherein said administration is by topical application.

17. The method of claim 15, wherein X¹ and X² are the same and are both tert-butyl and X⁰ is H.

18. The method of claim 17, wherein L is CH₂, B is H and t is 1.

19. The method of claim 18, wherein R¹, R², R³ and R⁴ are isopropyl.

20. The method of claim 15, wherein R¹, R², R³ and R⁴ are the same or different and are selected from hydrogen, methyl, ethyl, n-propyl, isopropyl, n-butyl, s-butyl and tert-butyl.

21. The method of claim 20, wherein R¹, R², R³ and R⁴ are identical but are not H.

22. The method of claim 21, wherein R¹, R², R³ and R⁴ are isopropyl.

23. The method of claim 15, wherein said diphosphonate of formula (I) is tetraisopropyl 2-(3,5-di-tert-butyl-4-hydroxyphenyl)-ethylidene-1,1-diphosphonate.

24. The method of claim 15, wherein said skin is postmenopausal skin.

25. The method of claim 15, wherein said skin is the skin of a subject with osteoporosis.

26. The method of claim 15, further comprising administration of an effective amount of an inhibitor of matrix metalloproteinase production or activity, a retinoid, an alpha hydroxy acid or derivative thereof, an anti-oxidant, a radical scavenging agent, an anti-inflammatory agent, or a mixture thereof.

27. A method for retarding and /or reversing the formation of fine lines and wrinkles in photoaged skin in a human subject requiring such treatment comprising the administration of an effective amount of a diphosphonate of formula (I): 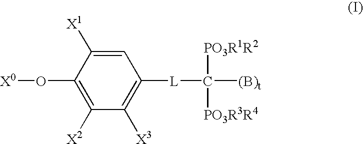
wherein X⁰ is H, an alkyl group having from 1 to 4 carbon atoms;
X¹, X² and X³ are identical or different and are H, a straight or branched alkyl or alkoxy group having from 1 to 8 carbon atoms,
R¹, R², R³ and R⁴ are identical or different and are H, a straight, branched or cyclic alkyl group comprising from 1 to 8 carbon atoms, or R¹, R², R³ and R⁴ may form an alkylidenedioxy ring comprising from 2 to 8 carbon atoms;
B is H, or an alkyl group having from 1 to 4 carbon atoms,
t is 0 or 1,
when t is 1 then L is —CH═CH—CH₂—, —(CH₂)ₙ—, —O(CH₂)ₙ—, —S—, —SO₂—, —S(CH₂)ₙ—, —SO₂ (CH₂)ₙ—, where n is an integer from 1 to 7;
or when t is 0 then L is —(CH═CH)ₖ —(CH₂)_{d} —CH═ where k is 0 or 1 and d is an integer from 0 to 4.

28. The method of claim 27, wherein said administration is by topical application.

29. The method of claim 27, wherein X¹ and X² are the same and are both tert-butyl and X⁰ is H.

30. The method of claim 29, wherein L is CH₂, B is H and t is 1.

31. The method of claim 30, wherein R¹, R², R³ and R⁴ are isopropyl.

32. The method of claim 27, wherein R¹, R², R³ and R⁴ are the same or different and are selected from hydrogen, methyl, ethyl, n-propyl, isopropyl, n-butyl, s-butyl and tert-butyl.

33. The method of claim 32, wherein R¹, R², R³ and R⁴ are identical but are not H.

34. The method of claim 33, wherein R¹, R², R³ and R⁴ are isopropyl.

35. The method of claim 27, wherein said diphosphonate of formula (I) is tetraisopropyl 2-(3,5-di-tert-butyl-4-hydroxyphenyl)-ethylidene-1,1-diphosphonate.

36. The method of claim 27, further comprising administration of an effective amount of an inhibitor of matrix metalloproteinase production or activity, a retinoid, an alpha hydroxy acid or derivative thereof, an anti-oxidant, a radical scavenging agent, an anti-inflammatory agent, or a mixture thereof.

37. A topical pharmaceutical composition comprising a pharmaceutically acceptable topical carrier and a compound of formula (I): 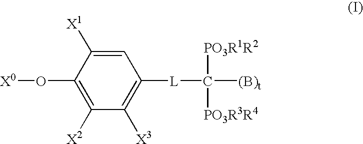
wherein X⁰ is H, an alkyl group having from 1 to 4 carbon atoms;
X, X² and X³ are identical or different and are H, a straight or branched alkyl or alkoxy group having from 1 to 8 carbon atoms,
R¹, R², R³ and R⁴ are identical or different and are H, a straight, branched or cyclic alkyl group comprising from 1 to 8 carbon atoms, or R¹, R², R³ and R⁴ may form an alkylidenedioxy ring comprising from 2 to 8 carbon atoms;
B is H, or an alkyl group having from 1 to 4 carbon atoms,
t is 0 or 1,
when t is 1 then L is —CH═CH—CH₂—, —(CH₂)ₙ—, —O(CH₂)ₙ—, —S—, —SO₂—, —S(CH₂)ₙ—, —SO₂(CH₂)ₙ—, where n is an integer from 1 to 7;
or when t is 0 then L is —(CH═CH)ₖ —(CH₂)_{d} —CH═ where k is 0 or 1 and d is an integer from
0 to 4;
wherein the said compound of formula (I) is in an amount effective to increase the expression of dermal collagen when applied to skin.

38. The composition of claim 37, wherein X¹ and X² are the same and are both tert-butyl and X⁰ is H.

39. The composition of claim 38, wherein L is CH₂, B is H and t is 1.

40. The composition of claim 38, wherein R¹, R², R³ and R⁴ are isopropyl.

41. The composition of claim 37, wherein R¹, R², R³ and R⁴ are the same or different and are selected from hydrogen, methyl, ethyl, n-propyl, isopropyl, n-butyl, s-butyl and tert-butyl.

42. The composition of claim 41, wherein R¹, R², R³ and R⁴ are identical but are not H.

43. The composition of claim 42, wherein R¹, R², R³ and R⁴ are isopropyl.

44. The composition of claim 37, wherein said diphosphonate of formula (I) is tetraisopropyl 2-(3,5-di-tert-butyl-4-hydroxyphenyl)-ethylidene-1,1-diphosphonate.

45. The composition of claim 37, further comprising an additional component selected from an inhibitor of matrix metalloproteinase production or activity, a retinoid, an alpha hydroxy acid or derivative thereof, an anti-oxidant, a radical scavenging agent, an anti-inflammatory agent, or a mixture thereof.
